(19) United States
(12) Patent Application Publication
(10) Pub. No.: US 2006/0242745 A1
(43) Pub. Date: Nov. 2, 2006

(54) Eyeglasses with replaceable lenses

(75) Inventor: Carmine DiChiara, Warren, NJ (US)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Liberty Sport, Inc.

(21) Appl. No.: 11/375,592

(22) Filed: Mar. 15, 2006

Related U.S. Application Data
(60) Provisional application No. 60/661,501, filed on Mar. 15, 2005.

(51) Int. Cl. A61F 9/00

(52) U.S. Cl. 2/12

(57) ABSTRACT

A type of eyewear has a pair of eyecup assemblies, one for each eye that sandwiches a lens between layers of resilient material and hard material to prevent the lenses from disengaging or popping out of the assembly. This sandwiching effect can be used in swim goggles or eyeglass frames including a chassis and a front retaining piece. Particularly relative to the swim goggles, the layers of each eyecup assembly include an eyecup, an inner eyecup retainer, and an outer cap releasably attached to the inner eyecup retainer. Each eyecup assembly has a nasal portion connected to a bridge strap and a temple portion connected to a head strap. The inside surface of the opening has an inner groove for receiving a lens, and the outside surface of the opening has an outer groove. With the eyeglass frames, the lenses are sandwiched between a chassis and a front retaining piece. A pin, screw, or clip further enhances this connection to secure the lenses, thus enabling the wear to resist impact.

### CROSS-REFERENCE TO RELATED APPLICATIONS

This application claims the benefit of U.S. Provisional Application 60/661,501, filed Mar. 15, 2005. That application is hereby incorporated by reference in its entirety.

### FIELD OF THE INVENTION

The present invention generally relates to eyeglasses with replaceable lenses. More particularly, the present invention relates to swim goggles with replaceable prescription lenses, or impact resistant glasses with replaceable prescription or plano (non-prescription) lenses.

### BACKGROUND OF THE INVENTION

Swim goggles are meant to protect the eyes from the water, look as streamlined and aesthetically pleasing as possible, and create as little drag as possible for the ardent swimmer.

Swimmers who need corrective lenses have generally had the option of either wearing swim goggles with piano (non-corrective) lenses, or wearing “Rx-able” swim goggles (that is, goggles with replaceable, prescription lenses). Current Rx-able swim goggles are generally unsatisfactory in that they either do not look good (due to the size and design required to accept a prescription lens), they are not totally watertight and allow water leakage, or they are too difficult to prescribe and therefore opticians refuse to work with them.

Conventionally, there are two options for mounting a prescription lens into current swim goggles. First, a prescription lens can be glued to the back of a pre-existing plano lens. This is not an easy operation, and therefore it is not very popular. Furthermore, there is a risk that that the prescription lens will hit the user's eye. Thus, the second option is more frequently used, and requires the use of either a soft or a hard eyecup to retain the lens. When using a hard eyecup, there is usually a leakage problem because it is difficult to make two hard surfaces completely water tight. This is typically corrected by applying a liquid sealer around the lens after insertion. This application risks contacting the liquid onto the lens surface and consequently, damaging the prescription lens optics. When using the soft eyecups, they must be thick enough to allow a lens to be inserted, and, after they are inserted, strong enough to retain the lens while in use. This requires the eyecup to be relatively thick, which is problematic.

With the rising awareness of the need for protective eyewear during sport activities, there is a problem with inserting a prescription lens into eyeglass frames while simultaneously meeting the impact standards set forth in the ASTM F803 and Military Ballistic standards. Conventionally, when working with high impact material, the only way to insert lenses is by mechanical means.

Accordingly, a need exists for providing improved eyewear, particularly swim goggles, with easily replaceable lenses. There is also a need for improved impact resistant frames with easily replaceable lenses.

### SUMMARY OF THE INVENTION

An object of the present invention is to provide aesthetically pleasing, yet functional, swim goggles.

Another object of the present invention is to provide swim goggles that allow easy replacement and insertion of lenses by an optician.

A further object of the present invention is to provide an easily insertable prescription lens for impact resistant frames while still having aesthetically pleasing frames.

Yet another object of the present invention is to provide prescription lenses sandwiched between an impact frame or goggle and a shield.

These objects are basically obtained by providing swim goggles including a pair of eyecup assemblies. Each eyecup assembly has a nasal portion, each of which is connected by a bridge strap, and a temple portion, each of which is connected to a head strap. The eyecup assembly includes an eyecup, an inner eyecup retainer, and an outer cap releasably attached to the inner eyecup retainer. The eyecup is formed of a resilient material and has an opening with an inner surface and an outer surface. The inner surface has an inner groove for receiving a lens, and the outer surface has an outer groove. The inner eyecup retainer is formed of a relatively rigid material. The inner eyecup retainer has an opening that fits around the outside edge of the eyecup. The inner surface of the opening in the retainer has a ridge that fits into the outer groove of the eyecup.

In a further aspect of the invention, a pair of impact resistant glasses includes a chassis having a pair of openings for receiving lenses. A retaining piece has a pair of openings that correspond to the openings in the chassis for mating with the chassis. The edges of the openings in the retaining piece and chassis are beveled or stepped. The beveled or stepped edges together form grooves for receiving the lenses.

In yet another aspect of the invention, similar to the previous aspect, a pair of retaining pieces mates with the chassis. Each retaining piece has a temple portion, a nose portion, and an opening that corresponds to one of the openings in the pair of openings in the chassis.

As used in this application, the terms “top”, “bottom”, and “side” are intended to facilitate the description of the eyewear, and are not intended to limit the eyewear of the present invention to any particular orientation.

Other objects, advantages, and salient feature of the invention will become apparent from the following detailed description, which, taken in conjunction with annexed drawings, discloses a preferred embodiment of the present invention.

### BRIEF DESCRIPTION OF THE DRAWINGS

The above and other objects, features, and advantages of certain embodiments of the present invention will be more apparent from the following description taken in conjunction with the accompanying drawings, in which:

FIG. 1 is an exploded, perspective view of a swim goggle according to a first embodiment of the present invention;

FIG. 2 is a sectional view of an assembled eye cup assembly of the swim goggle of FIG. 1;

FIG. 3 is an exploded, sectional view of the eye cup assembly of FIG. 2;

FIG. 4 is a front, elevational view of the swim goggles of FIG. 1;

FIG. 5 is a top, plan view of the swim goggles of FIG. 1;

FIG. 5a is a top, plan view of an enlarged section of the screw and bridge strap connection of the swim goggles of FIG. 5;

FIG. 6 is a left side view of swim goggles in FIG. 1 with the head strap retaining members;

FIG. 7 is a left side view of swim goggles of FIG. 1 without the head strap retaining members of FIG. 6;

FIG. 8 is a sectional view taken along line 8-8 in FIG. 4;

FIG. 9 is a front, elevational view of swim goggles in accordance with a second embodiment of the present invention;

FIG. 10 is a sectional view taken along line 10-10 in FIG. 9;

FIG. 11 is a sectional view taken along line 11-11 in FIG. 9;

FIG. 12 is a top, plan view of safety glasses according to a third embodiment of the present invention;

FIG. 13 is a front, elevational view of the safety glasses shown in FIG. 12;

FIG. 14 is a sectional view taken along line 14-14 in FIG. 13 with a v-shaped groove;

FIG. 15 is a sectional view taken along line 14-14 in FIG. 13 with a square shaped groove;

FIG. 16 is a front, elevational view of the retaining piece of the safety glasses shown in FIG. 12;

FIG. 17 is a front, elevational view of the chassis of the safety glasses shown in FIG. 12;

FIG. 18 is a left side view of the safety glasses shown in FIG. 12;

FIG. 19 is an expanded view of the connection portion of the chassis and the clip retaining assembly of the safety glasses shown in FIG. 12;

FIG. 20 is an expanded view of the connecting portion of the chassis and the screw retaining assembly of the safety glasses shown in FIG. 12;

FIG. 21 is a top, plan view of safety glasses according to a fourth embodiment of the present invention;

FIG. 22 is a front, elevational view of the safety glasses shown in FIG. 21;

FIG. 23 is a front, elevational view of the retaining pieces of the safety glasses shown in FIG. 21; and

FIG. 24 is a front, elevational view of the chassis of the safety glasses shown in FIG. 21.

Throughout the drawings, the same drawing reference numerals will be understood to refer to the same elements, features, and structures.

### DETAILED DESCRIPTION OF THE INVENTION

The matters defined in the description such as a detailed construction of the elements are provided to assist in a comprehensive understanding of the embodiments of the invention. Accordingly, descriptions of well-known functions and constructions are omitted for clarity and conciseness.

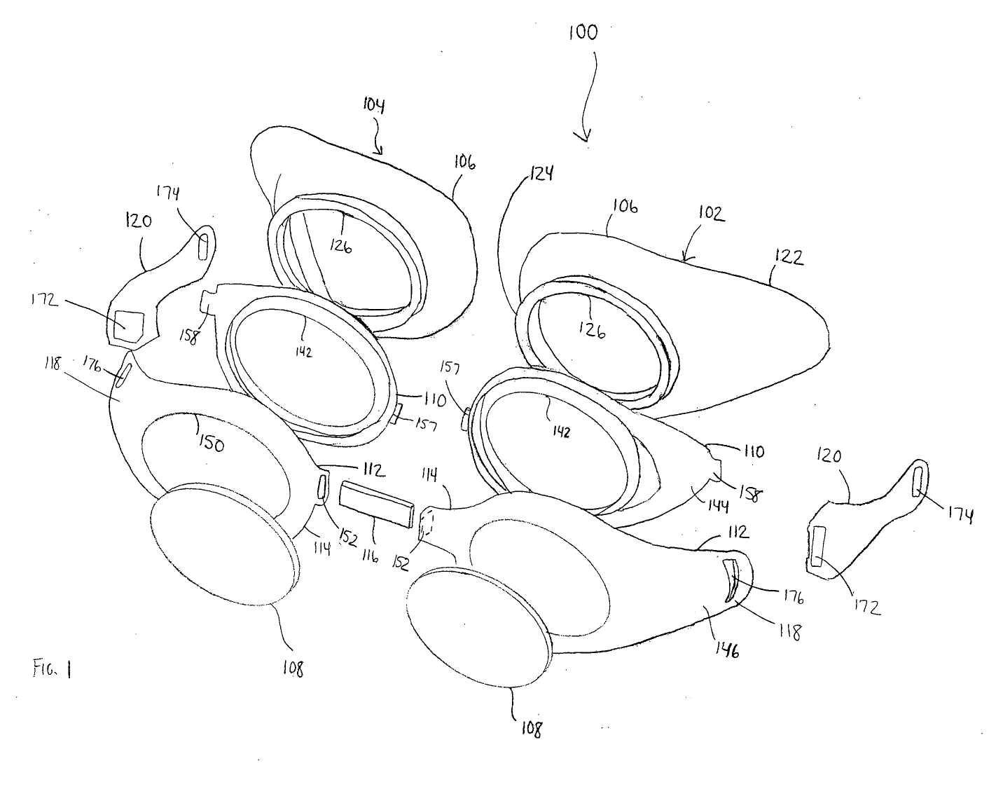

Referring to FIGS. 1 and 5a, a pair of swim goggles 100, or eyewear, has a left eyecup assembly 102 and a right eyecup assembly 104. Preferably, the left and right eyecup assemblies 102 and 104 are mirror images of one another. Each eyecup assembly 102, 104 has a soft eyecup 106, a lens 108, an inner eyecup retainer 110, and an outer cap 112. Each eyecup assembly 102, 104 has a nasal portion 114 for connecting to a bridge strap 116 which links the left and right eyecup assembly 102, 104. The nasal portions 114 of the left and right eyecup assemblies 102 and 104 that are connected by a bridge strap 116 can be secured to the bridge strap channel 152 by a screw 153 or similar type mechanism. Bridge strap 116 can also be snapped or glued into the bridge strap channel 152. The screw 153 engages the nasal portion 114 and the bridge strap 116 to connect the outer caps 112 to the bridge strap 116. The connection between the bridge strap 116 and eyecup assemblies 102, 104 is adjustable to fit over a wide variety of noses. Each eyecup assembly also has a temple portion 118 that may be attached, either directly or indirectly, to a head strap (not shown), thus forming a pair of swim goggles 100. The temple portion 118 stems horizontally from eyecup assemblies 102, 104 to guide the goggles 100 around a wearer's face.

Head strap retaining members 120 may be used to connect the head strap to the eyecup assemblies 102, 104. Head strap retaining members 120 each include a first slot 172 and a second slot 174. First slot 172 receives a head strap and threads the head strap horizontally through the first slot 172 and recess 176. An opposing end of the head strap is laced through the second slot 174. Head strap retaining members 120 prevent the head strap from detaching from the eyecup assemblies 102, 104, but also allow the size of the goggles 100 to be controlled because the retaining members 120 trap the head strap in a desired position as determined by the user. Head strap retaining members 120 are adjustable to secure the headstrap once the goggles 100 are attached to the user.

The soft eyecup 106 is formed of a resilient material, such as the material used for the eyecups in the AquaSpecs swim goggles available from Liberty Sport of Fairfield, New Jersey. Any suitable material which will create a substantially water tight seal when pressed against a swimmer's face can be used. Examples of suitable materials include rubber, rubber-type materials, leather, and foam. The resiliency enables the soft eyecup 106 to conform to the area surrounding each user's eyes. Once the soft eyecup 106 is compressed, it easily retains its form and springs back to its original compression. Thus, the goggles 100 can be used on many different shaped eye areas.

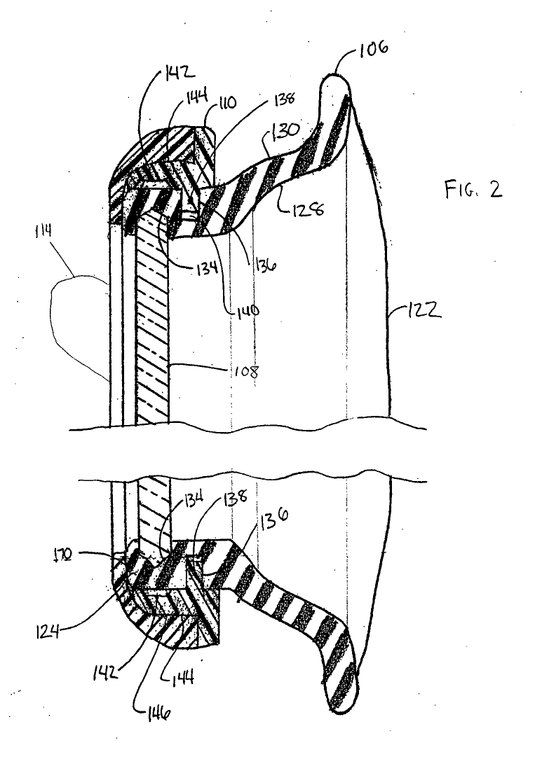

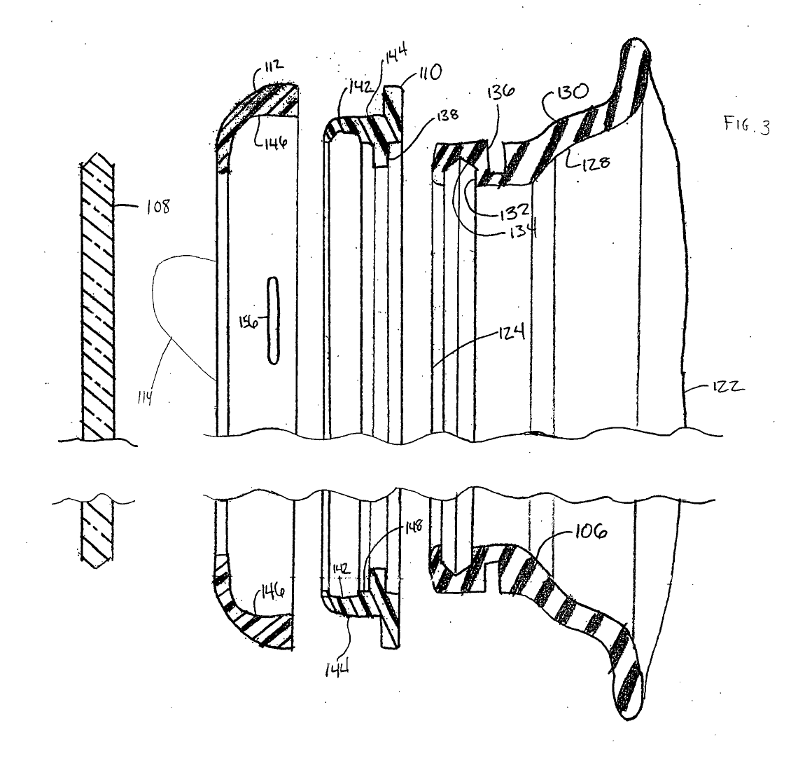

The rear portion 122 of the soft eyecup 106 is contoured so that it provides a comfortable seal against a swimmer's face, yet secure enough to prevent water from seeping into the eyecups 106. The front portion 124 of the soft eyecup 106 has an opening 126. As seen most clearly in FIGS. 2 and 3, the soft eyecup 106 has an inner surface 128 and an outer surface 130. The inner surface 128 attaches to the wearer's face and has a recess to allow the eyecup 106 to expand. An inner groove 132 is located on the inner surface 128 of the eyecup 106 near the opening 126. The inner groove 132 is generally perpendicular to the inner surface 128 junction. Preferably, the bottom surface 134 of the inner groove 132 is beveled to mate with the edge of the lens 108 but is not necessary as long as a seal is created. The bottom surface 134 should have the same shape on the upper section of the inner surface 128 and the lower section of the inner surface 128, but the lenses 108 do not need to be beveled, they can be square or substantially similar to a square shape. The bottom surface 134 behaves like a channel because it does not perfectly mate with the edge of the lens 108. There is some leeway between the edge of the lens 108, the bottom surface 134, and the inner groove 132 such that an eyewear specialist can replace the lenses 108, if necessary.

An outer groove 136 is located on the outer surface 130 of the soft eyecup 106. The outer groove 136 is sized to receive an extended ridge 138 located on the inner eyecup retainer 110. The outer groove 136 is generally a substantially rectangular shaped aperture for receiving a mating ridge 138. Preferably, a gap 140 is provided between the ridge 138 and the bottom of the groove 136 to provide some tolerance for easier assembly and to eliminate a vacuum effect when the mating ridge 138 and the groove 136 are engaged.

The inner eyecup retainer 110 is formed of a relatively rigid material, such as a plastic, or, more particularly, polycarbonates, propionates, or polypropylenes. This enables a secure and tight fit between the inner eyecup retainer 110 and the eyecup 106. The inner eyecup retainer 110 has a recess 142 sized to fit snugly around the outside surface 130 of the eyecup 106. Recess 142 also is shaped to provide a snug fit between the outer cap 112 and the inner eyecup retainer 110. The inner surface 148 of the inner eyecup retainer 110 has a ridge 138 and a recess 142.

The ridge 138 engages the outer groove 136 on the eyecup 106 to secure the inner eyecup retainer 110 into the eyecup 106. The recess 142 provides expansion space for the resilient eyecup 106 to expand when it is compressed during assembly, and the recess 142 assists in providing a tight seal. This connection enables a user to have secure goggles 100 while simultaneously allowing an eyewear professional to access the lenses 108 freely without having to pry it from the assembly. The contour of the outer surface 144 of the inner eyecup retainer 110 corresponds to the contour of the inner surface 146 of the outer cap 112 to provide a smoother and aesthetically appealing connection.

The outer cap 112 is formed of a relatively rigid material, such as a plastic. Optimal plastics may include polycarbonates and polypropylenes. The outer cap 112 has an opening 150, or first aperture, that is sized to fit over the inner eyecup retainer 110. The outer cap 112 is releasably attached to the inner eyecup retainer 110 with a corresponding recess 156 and a protrusion 157 located at the nasal portion of the inner eyecup retainer 110 and the outer cap 112. Additionally, as depicted in FIG. 8, the outer cap 112 is releasably attached to the inner eyecup retainer 110 at a junction with a connector located at the temple portions 118 and the outer cap 112. An inner border of the outer cap 112 presses against the outer surface to attach to the eyecups 102, 104.

As seen most clearly in FIGS. 5 and 5a, a bridge strap channel 152 is provided at the nasal portion 114 for receiving a soft bridge strap 116. The bridge strap 116 is preferably held into place by a screw 153 that transversely engages the bridge strap 116 and the bridge strap channel 152 to secure the bridge strap 116 to the nasal portion 114. Preferably, the screw 153 is stainless steel so that it is resistant to corrosion caused by saltwater. The bridge strap 116 may be made of any soft, flexible material, but comfortable to be worn across the user's nose for a substantial period of time while the user is engaging in sport activities.

Turning now to FIGS. 1-4 and 6, the outer cap 112, or first member, is fastened to the inner eyecup retainer 110, or second member, at both the nasal portion 114 and the temple portion 118 with a first connector and a second connector. At the opposite end of the outer cap 112, a recess 156 is provided on the inner surface 146 of the outer cap 112. The recess 156 is disposed to serve as the link to the eyecup retainer 110. The first connector includes a protrusion 157 formed on the outer surface 144 of the inner retainer 110, closest to the nasal area that fits into recess 156 disposed closest to the nasal portion 114 of the outer cap 112. Similarly, the second connector includes a protrusion 158 formed on the outer surface 144 of the inner retainer 110, closest to the temple area that fits into the recess 160 disposed closest to the temple portion 118 of the outer cap 112. The distance between the protrusions 157, 158 is greater than the distance between the recesses 156, 160 when the inner retainer 110 and the outer cap 112 are not engaged with each other and the inner retainer 110 is resilient.

The positions of the recesses 156, 160 and the protrusions 157, 158 could be reversed such that the recesses 156, 160 are on the outer surface 144 of the inner retainer 110 and the protrusions 157, 158 are disposed on the inner surface 146 of the outer cap 112. Therefore, when the goggles 100 are assembled, the connectors including the protrusions 157, 158 and the recesses 156, 160 hold the outer cap 112 into place with respect to the inner retainer 10. In other words, protrusions 157, 158 are both located on the outer surface 144 of the inner retainer 110, but at opposing ends. Recesses 156, 160 are both located on the inner surface 146 of the outer cap 112, but at opposing ends. Thus, from the connectors of the protrusions 157, 158 and the recesses 156, 160, this engagement creates the snapping relationship securing the outer cap 112 to the retainer 110.

Eyecup retainer 110 pivots to disengage from the outer cap 112. This disengagement is caused by protrusion 157 releasing from recess 156 at the nasal portion 114 of the goggles 100 and likewise, protrusion 158 releasing from recess 160 at the temple portion 118 of the goggles 100. This “sandwiching” effect traps the lenses 108 and prevents them from popping out of the goggles 100. There is a spring force, working together with the connectors, that locks in the lens 108.

The temple portion 118 of the outer cap 112 is designed to be connected to a head strap (not illustrated). The head strap is indirectly connected to the outer cap 112 by connecting to a head strap retaining member 120. The head strap retaining member 120 may be used to control the length of the head strap and adjust it for a proper fit. A slot is provided in the outer cap 112 for connection with the head strap retaining member 120. A head strap is laced through a head strap retaining member 120 and can be adjusted to suit a user's size. Head strap retaining member 120 is composed of a suitable material to support the adjustability of the head strap and the flexibility of eyecup assemblies 102, 104. Suitable strap attachment members are available from Liberty Sport with, for example, their Sting Ray model of swim goggles. By having both the bridge strap 116 and the head strap connected to the outer cap 112, in use, pressure is applied on the lens 108 so that it stays in place. Preferably, the head strap is a split strap to provide more positive positioning on a wearer's head.

The lens 108 of the present invention is preferably substantially planar. The outer edge of the lens 108 is preferably a v-shaped bevel since optical equipment is typically designed to construct v-shaped bevels, but nevertheless, the edge may be any suitable shape. The bevel is received in the groove 134 to engage the soft eyecup 106. Due to the lens 108 thickness, if necessary, the bevel connection could be stepped such that there is more than one level, thus allowing for multi-tiered lenses to be connected and snapped into the eyecup assembly 102, 104. This would depend on the required prescription or taper of the lenses.

The installation of a lens 108 into an eyecup assembly 102, 104 is as follows. First, the assembly of the left and right sides 102, 104 is disassembled, if it has been previously assembled. To do this, the outer cap 112 is flexed to unsnap the connectors of the protrusions 157, 158 and the recesses 156, 160. Protrusion 157 is disengages from 156 and then, protrusion 158 is disengaged from recess 160. The outer cap 112 is then set aside. A lens 108 of the desired prescription is then placed into the inner groove 132 in the soft eyecup 106. The recess 142, or second aperture, on the inner eyecup retainer 110 lets the soft eyecup material deform into the recess 142, and therefore eases the insertion of the lens 108. Furthermore, this helps to provide a watertight seal around the edge of the swim goggles 100 and the wearer's face. Once the lens 108 is in place, the outer cap 112 is replaced onto the inner retainer 110 and snapped back into place with the connectors. The inner surface 146 of the outer cap 112 presses against the front surface 170 of the eyecup 106, thereby helping to provide a better seal.

A second exemplary embodiment of the present invention is illustrated in FIG. 9. In this embodiment, each eyepiece assembly 300 is formed by a single, eyerim 302. The eyerim 302 is formed of a resilient material. It is substantially c-shaped because it has a split 304 in it where the open ends of the “c” face away from the bridge strap 316. The split 304 runs across the horizontal axis of the eyerim 302 and allows the eyerim 302 to be opened so that the outer cap 312 and lens 308 can be installed. A screw 306 made of metal, nylon, or any other suitable material allows a user to close the eyerim 302 and vertically engage the open ends of the eyerim 302. If desired, the screw 306 can be covered with a decorative cap.

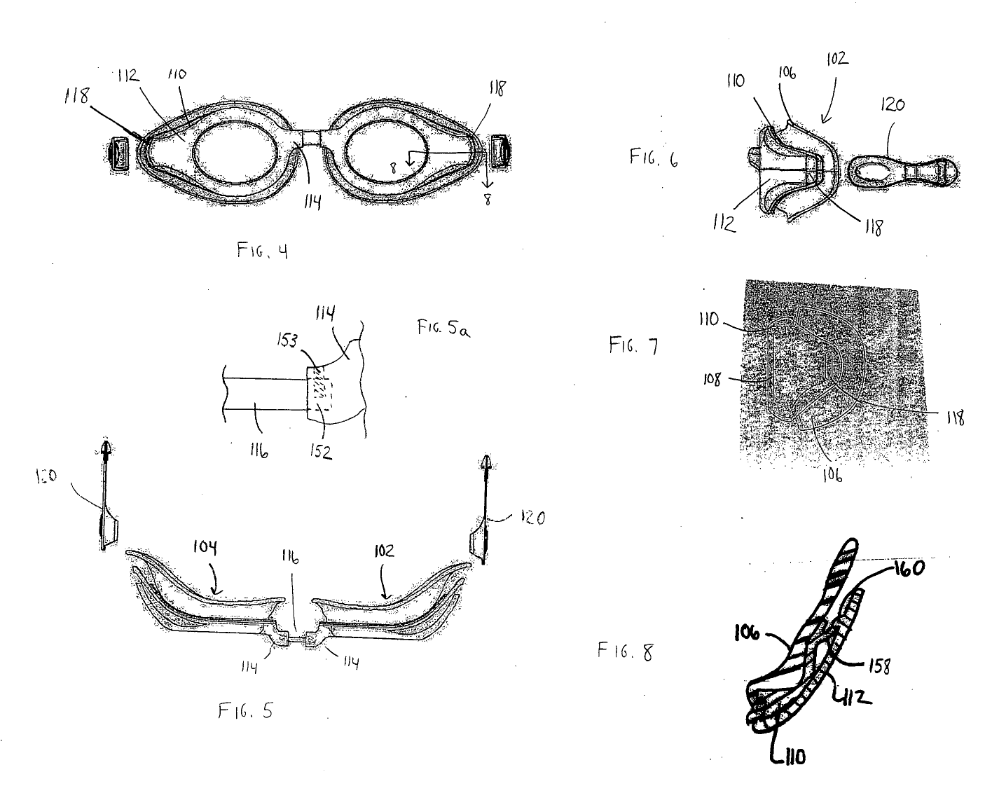

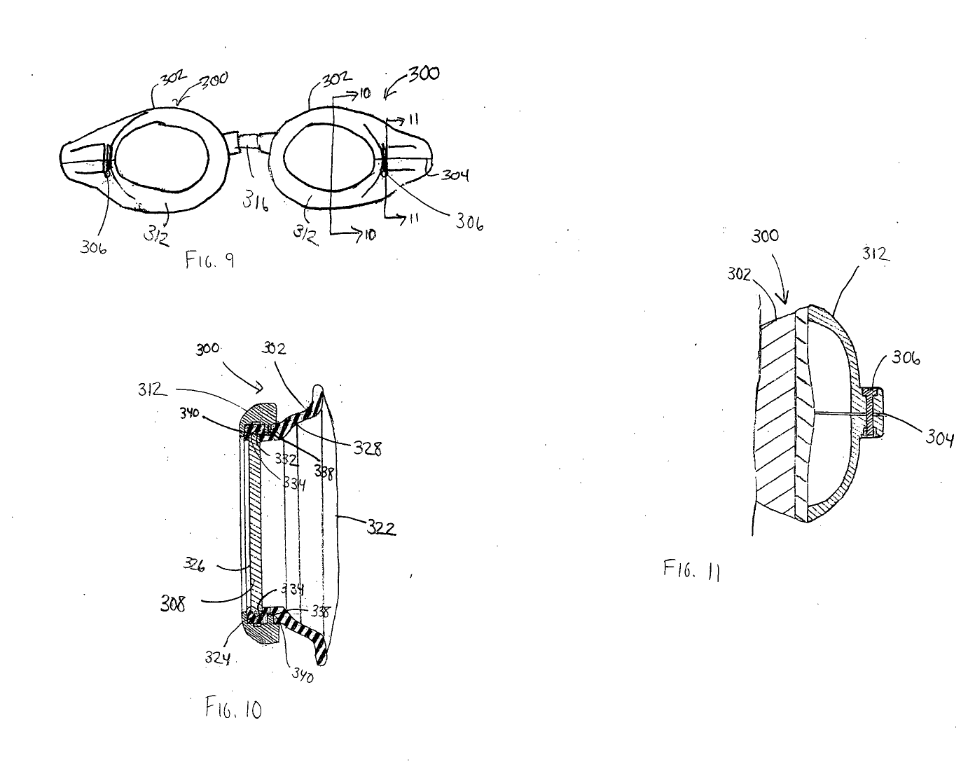

As illustrated in FIG. 10, the contour of the interior surface 328 of the eyerim 302 is substantially similar to the contour of the inner surface 128 of the assembled eyecup assembly 102, 104 of the previous embodiment. Accordingly, the operation and function of the eyerim 302 is substantially the same as already described with respect to the first embodiment. The eyerim 302 provides a comfortable seal against the swimmer's face, yet secure enough to prevent water from seeping into the rear portion 322 of the eyerim 302. The inner surface 328 attaches to the wearer's face.

The front portion 324 of the eyerim 302 has an opening 326 for receiving the lens 308. An inner groove 332 is located on the inner surface 328 near the opening 326. Preferably, the surfaces 334 of the inner groove 332 are beveled to mate with the edge of the lens 308. The eyerim 302 is sized to receive extended ridges 338 of the outer cap 312. These ridges 338 engage the eyerim 302 to snap around the lens 308 and secure the lenses 308 to the eyerim 302. The extended ridges 338 are generally substantially rectangular shaped projections for engaging apertures in the exterior surface of the eyerims 302. Preferably, a gap 340 is provided between the ridges 338 and the apertures to provide some tolerance for easier assembly and to eliminate a vacuum effect when the mating ridges 338 and the apertures of the eyerims 302 are engaged.

The outer cap 312 is formed of a relatively rigid material, such as a plastic, or, more particularly, polycarbonates, propionates, or polypropylenes. This enables a secure and tight fit between the eyerim 302 and the outer cap 312, thus protecting the lenses 308 from popping out of the eyepiece assembly 300.

FIG. 11 illustrates the screw 306 connection of the eyepiece assembly 300. Lens 308 is held in the eyepiece assembly 300. The rear portion of the soft eyerim 302 is contoured to provide a comfortable seal against a swimmer's face, yet secure enough to prevent water from seeping into the eyepiece assembly 300. The lens 308 is thus sandwiched between the different layers of the assembly 300, mainly the soft eyerim 302 and the outer cap 312. The pin connection 306 further secures this sandwiching and allows easy access to insert or extract the lens 308 from the eyepiece assembly 300, if desired.

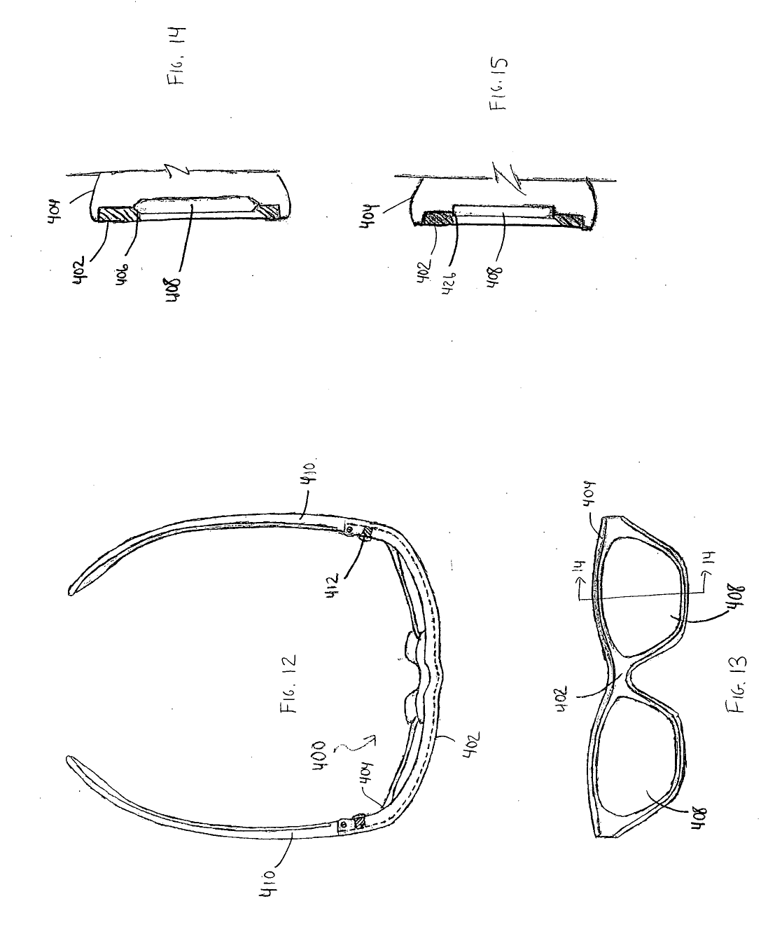

A third exemplary embodiment of the present invention is illustrated in FIGS. 12-20. In this embodiment, the concept of sandwiching a lens 408 between two hard pieces of an eyecup assembly 400, as described above, is used with standard frames 400, rather than swim goggles, as in the previous two embodiments. This provides another way to insert lenses 408 into a frame 400 that needs to meet impact standards.

In this embodiment, the glasses 400 have a front retaining piece 402 that is combined with a one piece chassis 404. When the front retaining piece 402 is combined with the chassis 404, the assembly forms a square-shaped 426 or a v-shaped groove 406 or openings for receiving a lens 408. The lens retainer 402 holds the lens 408 in place. An eyecup similar to that of the goggles 100, described in the first embodiment, can be inserted into the back of the chassis 404 and act as a seal and a guard against wind and debris. Half of the groove 406, 426 is formed on the front retaining piece 402 and half on the chassis 404.

The front retaining piece 402 is made from a rigid impact resistant material and shaped to create the front part of the glasses 400. The front retaining piece 402 is mated with the chassis 404 by snapping into the chassis 404 to lock the lenses 408 into place. The lenses 408 are sandwiched between the front retaining piece 402 and the chassis 404. The chassis 404 is also formed of a rigid impact resistant material. The edges are beveled or stepped and form grooves 406, 426, as seen in FIGS. 14, 15, to accept both the retaining piece 402 and to form the back half of the channel for receiving the lens 408.

Referring to FIG. 20, at the temple portions 410 of the glasses 400, pins or screws 412, are used to hold the front retaining piece 402 to the chassis 404 and insure the glasses 400 do not come apart upon impact. In this manner, lenses 408 may be easily inserted into and replaced in the glasses 400. Once the retaining piece 402 is attached to the chassis 404, the lenses are secured into the glasses 400. A pin or screw 412 engages the connection between the retaining piece 402 and the chassis 404 and fastens them together. The connection is towards the temple pieces 410 and away from the front of the glasses 400 to enhance the aesthetic quality of the frames. This particular orientation hides the connection from a front, elevational view of the glasses 400.

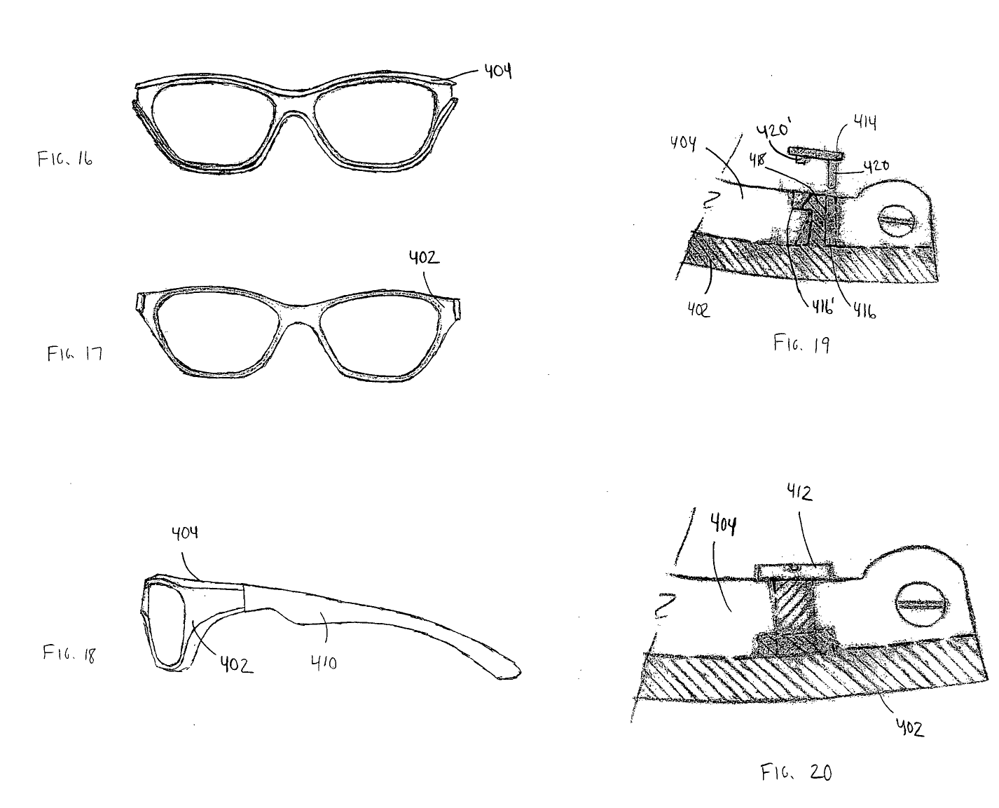

FIG. 19 illustrates another method of attaching the front retaining piece 402 to the chassis 404. Instead of the screw 412 illustrated in FIG. 20, a clip attachment including a lock pin 414 engaging the recesses 416, 416′ between the front retaining piece 402 and the chassis 404. Front retaining piece 402 further includes a protrusion 418. The hooks 420, 420′ of the lock pin 414 engage the area around the protrusion 418 to secure the connection between the front retaining piece 402 and the chassis 404. Recess 416 is engaged by hook 420 and recess 416′ is engaged by hook 420′. Hook 420′ is shaped to lie snugly against protrusion 418 for the upper section of the protrusion 418 can be angled towards the front retaining piece 402, sloping away from the inner section of the chassis 404. The hook 420′ has a mating shape that fits snugly against the slope of the protrusion 418. Similarly, recess 416 is substantially rectangular in shape and vertically disposed with respect to the protrusion 418. Recess 416 is engaged by hook 420 and further protects the front retaining piece 402 from disconnection with the chassis 404.

In the illustrated embodiment, the lens 408 is placed directly into the groove 406. If desired, a soft liner or an eyecup (not shown) can be used between the lenses 408 and the groove 406. The eyecup or liner will act as a cushion for the lenses 408 to be positioned and will help absorb any impacts. Additionally, it would provide extra protection around the eyes, which is ideal for a wearer engaged in sport activities.

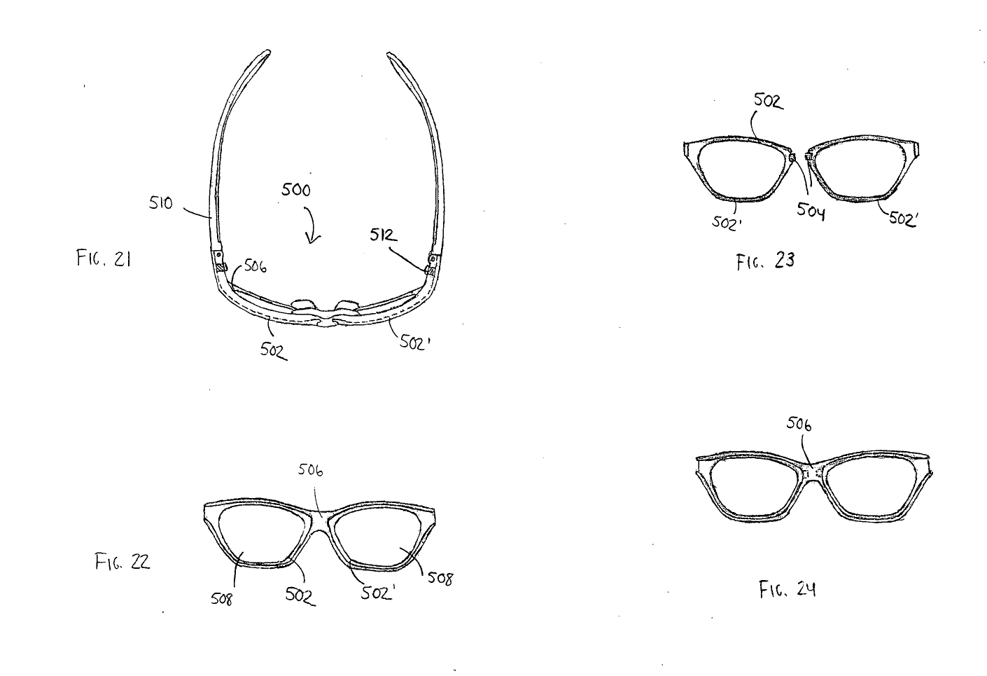

A fourth exemplary embodiment of the present invention is illustrated in FIGS. 21-24. The glasses 500 of this embodiment are similar to the embodiment shown in FIGS. 12-20 with the exception that two separate front retaining pieces 502, 502′ are provided, one for each lens. This provides yet another way of inserting lenses 508 into a frame 500 that needs to meet impact standards.

In this embodiment, the glasses 500 have two front retaining slots or pieces 502, 502′ combined with a one piece chassis 506 having two openings to receive lenses 508. When the front retaining pieces 502, 502′ are combined with the chassis 506, the assembly forms a square-shaped or a v-shaped groove for receiving a lens 508. The front retaining pieces 502, 502′ are made from a rigid impact resistant material and shaped to create the front part of the glasses 500. The front retaining pieces 502, 502′ are snapped into the chassis 506 to lock the lenses 508 into place. The lenses 508 are sandwiched between the front retaining pieces 502, 502′ and the chassis 506.

The front retaining pieces 502, 502′ are then connected to each other via the nose portions 504 with a connection assembly. The connection assembly is best described as a bridge (not shown). The bridge includes two outwardly opening retention slots to receive interlocking portions of the nose portions 504 of the front retaining pieces 502, 502′. The nose portions 504 could be glued or snapped into the bridge. In all other respects, this embodiment is substantially the same as previously described.

While various embodiments have been chosen to illustrate the invention, it will be understood by those skilled in the art that various changes and modifications can be made therein without departing from the scope of the invention as defined in the appended claims.

What is claimed is:

1. Eyewear comprising:
a pair of eyecup assemblies, each assembly having a nasal portion and a temple portion, and each assembly including an eyecup formed of a resilient material, said eyecup having an opening with an inner surface defined by an inner groove for receiving a lens, and an outer surface defined by an outer groove; and
an inner eyecup retainer sized to fit around an outside edge of said eyecup, formed of a relatively rigid material and having a ridge that fits into said outer groove of said eyecup.

2. Eyewear according to claim 1 wherein
an outer cap is releasably attached to said inner eyecup retainer and an inner border of said outer cap presses against said outer surface to attach to said eyecup.

3. Eyewear according to claim 1 wherein
a bridge connects said nasal portions of said eyecup assemblies.

4. Eyewear according to claim 1 wherein
a headstrap connects to each of said temple portions with each end of said headstrap.

5. Eyewear according to claim 4 wherein
at least two strap attachment members engage said headstrap for tightening said headstrap to said temple portions.

6. Eyewear according to claim 2 wherein
said outer cap is releasably attached to said inner eyecup retainer with a first recess and a first protrusion located at said nasal portion of said inner eyecup retainer and said outer cap, and a connector located at said temple portions and said outer cap.

7. Eyewear according to claim 6 wherein
said connector includes a second recess and a second protrusion.

8. Eyewear according to claim 1 wherein
a substantially planar lens is positioned in said inner groove.

9. Eyewear according to claim 8 wherein
said lens includes a beveled perimeter.

10. Eyewear according to claim 9 wherein
said beveled perimeter is substantially v-shaped.

11. Eyewear according to claim 1 wherein
an inner surface of said inner eyecup retainer has a recess to allow said eyecup to expand.

12. Eyewear for swimming, comprising:
an eyecup formed of a resilient material, said eyecup having an opening with an inner surface and an outer surface, said inner surface having an inner groove for receiving a lens, and said outer surface having an outer groove; and
an inner eyecup retainer formed of a relatively rigid material, said inner eyecup retainer sized to fit around an outside edge of said eyecup and having a ridge that fits into said outer groove.

13. Eyewear according to claim 12 wherein
an outer cap is releasably attached to said inner eyecup retainer.

14. Eyewear according to claim 13 wherein
said outer cap is releasably attached to said inner eyecup retainer with a corresponding recess and a protrusion located at said nasal portion of said inner eyecup retainer and said outer cap, and a connector located at a temple portion of said inner eyecup retainer and said outer cap.

15. Eyewear according to claim 13 wherein
a substantially planar lens is placed in said inner groove.

16. Eyewear according to claim 15 wherein
said lens includes an outside beveled perimeter.

17. Eyewear according to claim 16 wherein
said beveled perimeter is substantially v-shaped.

18. Eyewear according to claim 12 wherein
an inner surface of said inner eyecup retainer further includes a recess to allow said eyecup to expand.

19. Eyewear according to claim 13 wherein
an inner border of said outer cap presses against said outer surface.

20. Eyewear for swimming goggles comprising:
an eyerim formed of a resilient material, said eyerim having an opening with an inner surface and an outer surface, said inner surface having an inner groove for receiving a lens, and said outer surface having an outer groove, said eyerim is substantially c-shaped with a split portion;
an eyecup retainer formed of a relatively rigid material, said eyecup retainer sized to fit around said outer surface of said eyecup and having a ridge that fits into said outer groove within said split portion; and
a means for closing said split portion connected therein.

21. Eyewear according to claim 20 wherein
said means for closing said split portion is a screw.

22. Eyewear according to claim 20 wherein
said means for closing said split portion is a clip attachment.

23. Eyewear comprising:
a chassis having a pair of chassis openings for receiving a lens, said chassis openings including a beveled or stepped edge; and
a retaining piece mated with said chassis, said retaining piece including a pair of retaining openings corresponding to said chassis openings, said chassis and said retaining openings including a plurality of beveled or stepped edges, wherein said edges form grooves for receiving a lens.

24. Eyewear according to claim 23 wherein
one of a pin, a screw, and a snap fastens said chassis to said retaining piece.

25. Eyewear according to claim 23 wherein
said chassis and said retaining piece are connected by a plurality of a pin, a screw, and a snap.

26. Eyewear comprising:
a chassis having a first and a second chassis opening;
a first and a second retaining piece mated with said first and second chassis opening, said first and second retaining pieces each having a nose portion connected with a bridge; and
a lens sandwiched between said chassis and said first and second retaining pieces.

27. Eyewear according to claim 26 wherein
at least one of a screw, a snap, and a pin fastens said nose portions to said chassis.

28. Eyewear according to claim 26 wherein
said nose portions and said chassis are fastened with a plurality of a screw, a snap, and a pin.

29. Eyewear comprising:
a first member having a nasal portion, a temple portion, and a first aperture;
a second member having a nasal area, a temple area, and a second aperture;
a lens received between said first and second members;
a first connector located at said nasal portion and said nasal area and including a first recess disposed on one of said nasal portion and said nasal area and a first protrusion disposed on the other of said nasal portion and said nasal area; and
a second connector located at said temple portion and said temple area and including a second protrusion disposed on one of said temple portion and said temple area and a second recess disposed on the other of said temple portion and said temple area.

30. Eyewear according to claim 30, wherein
said first protrusion is disposed on said nasal portion and engages said first recess disposed on said nasal area.

31. Eyewear according to claim 31, wherein
said second protrusion is disposed on said temple portion and engages said second recess disposed on said temple area.

32. Eyewear according to claim 29, wherein
said second member is resilient, and the distance between said first and second protrusions is greater than the distance between said first and second recesses when said second member is not engaged with said first member.
